(19) United States
(12) Patent Application Publication
(10) Pub. No.: US 2010/0016421 A1
(43) Pub. Date: Jan. 21, 2010

(54) METHODS FOR DETERMINING SENSITIVITY TO AMINOFLAVONES

(76) Inventors: Angelika M. Burger, Detroit, MI (US); Binh Nguyen, Indianapolis, IN (US)

Correspondence Address: DICKSTEIN SHAPIRO LLP, 1825 EYE STREET NW, Washington, DC, 20006-5403, US

(21) Appl. No.: 12/485,695

(22) Filed: Jun. 16, 2009

Related U.S. Application Data
(60) Provisional application No. 61/129,280, filed on Jun. 16, 2008.

(51) Int. Cl. A61K 31/352

(52) U.S. Cl. 514/456

(57) ABSTRACT

Methods and compositions for treating cancer are discussed herein. Specifically, methods for determining the sensitivity of a patient to treatment with therapeutic agents, compositions for treating patients, and the treatment methods thereof are provided.

This application claims the benefit of U.S. provisional patent application Ser. No. 61/129,280 entitled “Methods for Determining Sensitivity to Aminoflavones” filed on Jun. 16, 2008, which is hereby incorporated by reference in its entirety.

### BACKGROUND

All publications and patents cited herein are incorporated by reference in their entirety.

Flavonoids, either natural or synthetic, exhibit a variety of biological activities. Such compounds, for example, inhibit protein kinase C, aromatase, topoisomerase, or cyclin-dependent kinase activity or exhibit antimitotic activity. In particular, 5,4′-diaminoflavones exhibit cytotoxicity against the human breast cancer cell line MCF-7. See Akama et al., J. Med. Chem., 41, 2056-2067 (1998).

Experiments incorporating various substituent groups at the 6, 7, 8, and 3′-positions on the flavone ring yielded some speculation as to the structure-activity relationship of the substituents, particularly at the 7-position of 5,4′-diamino-6,8,3′-trifluoroflavone. Certain physical properties of the parent flavone compound, such as solubility, could be improved by the presence of some, but not all, substituent groups at the 7-position. Certain 7-substituted compounds also demonstrated cytotoxicity against certain human breast cancer cells. See Akama et al., J. Med. Chem., at 2061-62.

Derivatives of aminoflavones exhibit growth inhibiting properties (see e.g., U.S. Pat. Nos. 5,539,112 and 6,812,246) against certain breast tumors, while other breast tumors are resistant (i.e., have limited biological response) to aminoflavones. Recently, the National Cancer Institute performed a human tumor screen (“the NCI 60 screen”) for various tumor cell lines to examine the sensitivity of the cell lines to aminoflavone. The NCI 60 screen measures the ability of a compound to selectively kill or inhibit the growth of diverse human cancers. The results of the NCI 60 screen showed generally that aminoflavone (AF) and its pro-drug AFP464 were active against certain types of human solid tumors (e.g., non-small cell lung cancer, renal cancer, and melanoma). See U.S. Pat. No. 6,812,246 at FIGS. 1A-1D; see also Meng et al., Activation of Aminoflavone (NSC 686288) by a Sulfotransferase is Required for the Antiproliferative effect of the Drug and for Induction of Histone γ-H2AX, Cancer Research 2006; 66: (19), 9656-64 (Oct. 1, 2006).

A total of 8 cell lines for breast cancer were screened in the NCI 60 screen (see U.S. Pat. No. 6,812,246 at FIG. 1B). However, of the 8 cell lines screened, 2 cell lines were sensitive to aminoflavone while 6 were resistant to aminoflavone. See U.S. Pat. No. 6,812,246 at FIG. 1B. The apparent discrepancy was attributed to the presence or absence of an estrogen receptor (“ER”). The two sensitive cell lines are ER-positive, while the six resistant cell lines are ER-negative. Importantly, the ER-positive cell lines are sensitive to AFP464 in the order of magnitude of 10 nanomolar, whereas the ER-negative cell lines appear resistant to AFP464 with an GI/IC50 as high as between 10 and 100 micromolar. In this case, a cell line that is sensitive to AFP464 has an IC50 value with respect to AFP464 of less than 1 μM whereas a resistant cell line will have an IC50 value with respect to AFP464 of greater than 1 μM.

Thus, while ER-positive tumors are sensitive to aminoflavones, ER-negative tumors did not appear to be sensitive to aminoflavones.

### BRIEF DESCRIPTION OF THE DRAWINGS

FIG. 1A shows localization of aryl hydrocarbon receptors (AhR) in select breast cancer cell lines, that AhR is predominantly localized in the cytoplasm of AF-sensitive MCF-7 and MCF-TAM1 breast cancer cell lines and localized in the nuclei of AF-resistant MDA-MB-231 and Hs578T breast cancer cell lines;

FIG. 1B shows immunofluorescence of phosphorylated γ-H2AX (FIG. 1B), a marker for DNA double strand breaks, indicating that the AF:AhR complex had translocated from the cytoplasm to the nucleus to activate a signaling cascade ultimately leading to DNA-damage and cell death;

FIG. 2 is an immunofluorescence stain of AhR in select ovarian cancer cell lines, and shows that AhR is predominantly localized in the cytoplasm of AF-sensitive OVCAR-3 ovarian cancer cell line, and localized in the nucleus of AF-resistant OVCAR-8 ovarian cancer cell line;

FIG. 3 is an immunofluorescence staining of AhR in select “triple negative” breast cancer cell lines and shows that MX-1 cells with nuclear AhR are resistant to treatment with AFP464 (IC50=30 μM), whereas the HCC1937 and IGROV1 lines with cytoplasmic AhR are sensitive to AFP464 (IC50s 10-200 nM), and also shows the growth curves for all three cell lines resulting from 5 day continuous exposure of the cells to AFP464 in 96-well plates and the detection of cell growth by conversion of methyltetrazolium into formazan by viable cells (MTT assay);

FIGS. 4A and 4B are Western blots showing ER expression after pretreatment with a histone deacetylase (HDAC) inhibitor, and show that upregulation of ER expression is correlated with sensitivity of cells to aminoflavone (IC50 of ˜1 μM);

FIG. 5 is a bar graph of CYP1A1 and CYP1B1 expression in “triple negative” breast cancer cell lines pretreated with a histone deacetylase (HDAC) inhibitors followed by treatment with AFP464 (if applicable), and show that the expression of CYP1A1 and CYP1B1 is upregulated when treated with SAHA (suberoylanilide hydroxamic acid) followed by treatment with AFP464; and

FIG. 6 shows a line graph illustrating data collected from in vivo studies using a “triple negative” breast cancer xenografts indicating that pretreatment of tumors with SAHA followed by treatment with AFP464 results in decreased cell growth compared to the use of AFP464 alone.

### DETAILED DESCRIPTION

Described herein are methods of determining sensitivity to aminoflavone compounds and administering aminoflavone compounds to a patient in need of treatment.

The terms “tumor,” “tumor cell,” “cancer” and “cancer cell” all refer to cells that exhibit abnormal growth, characterized by unregulated proliferation with or without loss of differentiation. The terms “tumor,” “tumor cell,” “cancer” and “cancer cell” include metastatic as well as non-metastatic cancer.

“Treatment of,” “treatment,” or “treating” cancer refers to an approach that provides beneficial or desired clinical results, including limiting progression of, stabilization or regression of tumor cells. Beneficial or desired clinical results include, but are not restricted to, alleviation or amelioration of one or more symptoms or conditions, diminishment of extent of disease, stabilization of the state of disease, prevention of development of disease, prevention of spread of disease, delay or slowing of disease progression, delay or slowing of disease onset, amelioration or palliation of the disease state, and remission (whether partial or total). “Treatment of,” “treatment,” or “treating” can also mean prolonging survival of a patient beyond that expected in the absence of treatment. “Treatment of,” “treatment,” or “treating” can also mean inhibiting the progression of disease temporarily and/or halting the progression of disease permanently in a subject. “Treatment of” “treatment,” or “treating” can also refer to an approach that arrests the growth of, kills, restores normal growth to, or slows the growth of a tumor, tumor cell, or cancer cell or groups of tumor cells or cancer cells. “Treatment of,” “treatment,” or “treating” can also refer to prescribing or administering a compound to a mammal to provide beneficial or desired clinical results, including stop of progression, stabilization or regression.

The term “administer” or “administering” refers to providing, prescribing, injecting, ingesting, or any other method or means of obtaining and/or introducing a therapeutic compound or medical treatment to a patient in need of such treatment.

The following detailed description provides sufficient detail to enable those skilled in the art to practice the claimed invention, and it is to be understood that other examples may be employed, and that modifications and substitutions may be made.

In one case, a method for treating a tumor in an animal is provided. For example, a tumor can be analyzed (e.g., by biopsy tissue sample, analysis of a marker for the tumor, or analysis of circulating tumor cells) to determine if the tumor has a predetermined gene cluster (e.g., luminal or basal A type) and if so, aminoflavone compounds can be administered to the patient. In another case, if the tumor has basal B type of gene cluster, then the patient is pre-treated with an agent capable of modifying gene transcription, such as, for example, histone deacetylase (HDAC) inhibitors before treatment with aminoflavone compounds.

The method of determining the genetic profile optionally includes excising tumor cells from a patient, washing, and optionally disassociating, and resuspending the cells in for example, phosphate buffered saline (PBS). Total RNA is then extracted from these cells and subjected to whole genome analysis using either Affymetrix or Illumina human genome arrays.

Breast tumor cells, either disassociated or not, can be categorized based on their genetic profiles and histology. For example, Neve and colleagues have categorized certain breast cancer cell lines by their genetic and histological profiles. See Neve R. M., et al., A collection of breast cancer cell lines for the study of functionally distinct cancer subtypes, Cancer Cell, December 2006; 10(6):515-27. These genetic and histological profiles can be performed on a variety of cancer cells e.g, ovarian, colon, prostate, bone, liver, lung, small intestine, pancreas, and skin.

The ER status of the collected cells can be determined by methods known in the art (e.g., immunofluorescence or immuno peroxidase assay, Western blot analysis, identification of ER-specific mRNA, and interaction of ER-ERE complexes with three different ER-specific antibodies measured by electrophoresis mobility assay (EMSA)). See, e.g., Averboukh et al. Classification of Breast Cancer Cells on the Basis of a Functional Assay for Estrogen, Molecular Medicine, Vo. 4, Issue 7, 454-467 (July 1998). For example, an antibody specific to ER-alpha, such as, for example, TE1-11 (sold by Abcam (Cambridge, Mass.) as ab16460) or Cell Signaling Technology, Danvers, Mass. (#2512 ) could be used in conjunction with an anti-mouse antibody conjugated with FITC, to perform and immunofluorescence assay or with horseradish peroxidase to perform immunohistochemistry to detect the estrogen receptor.

The luminal or basal status can be determined by methods known in the art. For example, prediction analysis of microassays (PAM) in accordance with Tibshirani et al., Diagnosis of Multiple Cancer Types by Shrunken Centroids of Gene Expression. Proc Natl Acad Sci USA 99:6567-6572 (2002), can be used as could the Human Genome U133A 2.0 Genechip® Assay (by Affymetrix®). These luminal and basal statuses can be further subdivided into luminal A and luminal B and basal A and basal B by the same methods discussed above.

For example, gene expression profiles using known markers for the genes ERBB3-, ESR1-positive, ESR1-negative, CAV1-positive, KRT5-, KRT14- can be determined by the Human Genome U133A 2.0 Genechip® Assay (by Affymetrix®). Luminal cell type breast cancer cells typically overexpress the ERBB3- and ESR1-positive genes when compared to basal cell type breast cancer cells. Likewise, basal cell type breast cancer cells overexpress the ESR1-negative and CAV1-positive genes when compared to f luminal cell type. The basal cell type can be further subdivided into basal A and basal B cell types. Basal A cell types overexpress the KRT5- and KRT14-positive genes when compared to basal B cell types. Basal B cell types have higher expression of VIM-positive genes than basal A. The term “overexpress” or “overexpression” refers to an increase in mRNA and/or copy number of a gene or gene cluster in a cell suspected to be tumorigenic by at least about 2-fold over a cell that is of a different genotype, normal or not considered to be tumorigenic. Overexpression can be determined, for example, by comparing the actual mRNA levels, copy number of a gene or gene cluster using nucleic acid probes or similar techniques or, for example, by measuring the intensity of the binding of an antibody to the protein or proteins encoded by the gene or gene cluster using immunofluroscence or radiolabeled antibodies or similar detection reagents.

In another case, breast cancer patients who have previously failed up to two prior chemotherapeutic regimens or ER-positive patients who have failed hormonal treatment, are treated first with Vorinostat (or suberoylanilide hydroxamic acid (SAHA)), an HDAC inhibitor, prior to treatment with AFP464. The HDAC inhibitor can be administered at a dose of about 1 to about 400 mg/kg, preferably at a dose of about 200 to about 400 mg daily. The HDAC inhibitor can be administered 2 to 7 days prior to the administration of AFP464, preferably 3 to 6 days prior to the administration of AFP464, and most preferably 5 days prior to the administration of AFP464.

AFP464 is preferably administered at a dose of about 1 to about 100 mg/kg, preferably about 35 to about 70 mg/kg, and most preferably about 35 mg/kg. A treatment schedule may include at least one additional dose of AFP464 at any dosage range discussed above. Additional AFP464 doses can be administered on any day after the initial dose, preferably on days 3, 5, 15, 17, and 19 (counting day 1 as the initial dose day).

At least one additional dose of HDAC inhibitor could also be administered before the AFP464 administration. For example, HDAC inhibitor can be administered 3 days and again 1 day prior to the AFP464 administration. The additional dose of HDAC inhibitor can be administered after the AFP464 administration as well (in addition to or in place of the additional dose prior to the AFP464 administration). For example, the additional dose of HDAC can be administered 10 to 15 days after the AFP464 administration, preferably 12-14 days after the AFP464 administration. HDAC inhibitors can be administered 12, 13, and 14 days after the AFP464 administration as well.

Examples of aminoflavone compounds that can be used in accordance with the description above include the following:

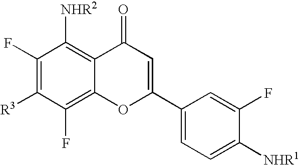

wherein each of R¹ and R² is H, COCH₂—R⁷, wherein R⁷ is amino, branched or straight-chain alkylamino, dialkylamino, or alkyl- or dialkylaminoalkyl, or an α-amino acid residue, provided that at least one of R¹ and R² is other than H, and

wherein R³ is H, branched or straight-chain alkyl, hydroxyalkyl, alkanoyloxyalkyl, alkanoyloxy, alkoxy, or alkoxyalkyl, or

a pharmaceutically acceptable salt thereof.

Other examples include 5-amino-6,8-difluoro-2-[3-fluoro-4-[(L-lysyl)amino)]phenyl]-7-methyl-4H-1-benzopyran-4-one, 5-amino-2-[4-[2-amino-5-guanidinopentanoyl]amino]-3-fluorophenyl]-6,8-difluoro-7-methyl-4H-1-benzopyran-4-one, 6,8-difluoro-7-methyl-5-(dimethylamino)acetamido-2-[4-(dimethylamino)acetamido-3-fluorophenyl]-4H-1-benzopyran-4-one, and 5-amino-6,8-difluoro-7-methyl-2-[4-(dimethylamino)acetamido-3-fluorophenyl]-4H-1-benzopyran-4-one.

An aminoflavone compound that can be used as described above includes AFP464, which has the following structure:

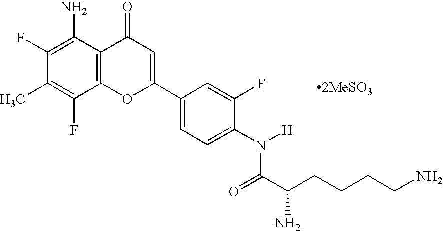

Without being limiting, aminoflavone compounds (and their derivatives, pharmaceutically acceptable salts, or substitutions) that can be used in accordance with the disclosure herein include any compound shown in Table 1.

TABLE 1
Representative Structures of Novel Aminoflavone
Compounds

| | |
|---|---|
| | (I) |
| 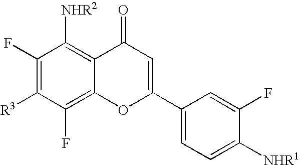 | |

| Cpd | R¹ | R² | R³ |
|---|---|---|---|
| 1a | CO—CHNH₂—(CH₂)₄—NH₂ | H | CH₃ |
| 1b | CO—CHNH₂—(CH₂)₃—NH—C(NH)—NH₂ | H | CH₃ |
| 1c | CO—CH₂—N(CH₃)₂ | H | CH₃ |
| 1d | CO—CH₂—N(CH₃)₂ | CO—CH₂—N(CH₃)₂ | CH₃ |
| 1e | CO—CH₂—NH₂ | H | CH₂OH |
| 1f | CO—CH₂—N(CH₃)₂ | H | CH₂OAC |
| 1g | CO—CH₂—N(CH₃)₂ | H | CH₂OH |
| 1h | CO—(CH₂)₂—N(CH₃)₂ | H | CH₂OAC |
| 1i | CO—(CH₂)₂—N(CH₃)₂ | H | CH₂OH |
| 1j | CO—(CH₂)₃—N(CH₃)₂ | H | CH₂OAC |
| 1k | CO—(CH₂)₃—N(CH₃)₂ | H | CH₂OH |
| 1l | H | CO—CH₂—NH₂ | CH₂OH |
| 1m | H | CO—CH₂—N(CH₃)₂ | CH₂OH |
| 1n | H | CO—(CH₂)₂—N(CH₃)₂ | CH₂OH |

The compounds and compositions as described herein may be formulated in a form suitable for any route of administration (e.g., oral, subcutaneous, and parenteral administration). Parenteral administration includes, for example, intravenous, intraperitoneal, intrapulmonary, and intrathecal. The compound and composition could also be administered topically

Additionally, screening test kits can also be made and sold to hospitals, nurses, doctors, and/or other healthcare professionals. Typically, a pathologist can take a biopsy of the breast tumor tissue, and determine the gene profile of the tumor cells contained in the tumor tissue to determine whether the tumor would be sensitive to aminoflavone (or any other aminoflavone analog or prodrug) by any of the methods discussed above. The healthcare provider may then administer aminoflavone to treat the tumor if the gene expression profile indicates sensitivity to aminoflavone. A test kit in accordance with this disclosure can include materials needed for determining gene expression and copy number (e.g., nucleic acid probes and reagents or microarray chip). Alternatively, based on the genetic profile of the patient, for example, basal B, the healthcare provider may administer a HDAC inhibitor prior to or in combination with an aminoflavone (e.g., AFP464).

Screening test kits can also include the immunohistochemistry or immunofluorescence staining of AhR or immunostaining with quantum dot technology of tumor biopsy samples or circulating tumor cells to determine the localization of AhR with known antibodies to AhR.

In another case, methods of reducing tumor volume comprising the methods disclosed herein can reduce tumor volume by 15% to 85% as compared to untreated tumors. Preferably, the tumor volume can be reduced by 25% to 75% by methods disclosed herein as compared to untreated tumors, and most preferably by 59%.

In yet another case, a method for inhibiting and/or reducing the growth of a tumor (e. g. breast, renal, ovarian or pancreatic) in an animal is provided. AFP464 complexes with AhR in the cytoplasm of the cells; the AFP464:AhR complex translocates to the nucleus to induce CYP1A1 transcription. CYP1A1 leads to a cascade pathway ultimately resulting in cell death. Therefore, the tumor or circulating tumor cells can be analyzed to determine if the AhR is predominantly localized to the cytoplasm of the tumor cells, and if so, aminoflavone compounds can be administered to the patient. The localization of AhR in a tumor can be determined by, for example, immunohistochemistry or immunofluorescence staining of a sample of the tumor or circulating tumor cells with an antibody directed to AhR. For example, antibodies sold by Biomol International/Enzo Life Sciences, Plymouth Meeting, Pa., USA (#SA-210) or Abnova (Taiwan) under catalog number H00000196-M02 can be used to determine the localization of AhR. The localization of aryl hydrocarbon receptors (AhR) can be determined by methods known in the art. For example, the localization can be determined by incubating the disassociated cancer cells in suspension with antibodies to AhR that are commercially available (Abnova, Biomol), and detecting the AhR by immunofluorescence or by using archival tumor tissue and detecting AhR by immunoperoxidase based histology.

These and other properties and advantages of the present disclosure will be apparent to one of ordinary skill in the art upon reading the detailed description. It is to be understood that application of the disclosure to a specific problem or environment will be within the capability of one having ordinary skill in the art. Some of the implementations of the disclosure are illustrated by the following non-limiting examples.

The breast cancer cell lines shown in Table 2 were screened to determine the localization of the aryl hydrocarbon receptor (AhR) in accordance with the techniques discussed above. MTT assays were conducted to measure cell death on various breast cancer cell lines. In this example, breast cancer cell lines having IC50 and IC 100 values less than 1 μM after treatment with AFP464 were deemed AF-sensitive; those breast cancer cell lines having IC50 and IC 100 values greater than 1 μM after treatment with AFP464 were deemed AF-resistant. All cell lines where the AhR is predominantly in the cytoplasm were shown to be AF-senstive. MCF-7 had IC50 and IC100 values of 16 nM and 300 nM, respectively. MCF-7 HER2-18 (MCF-7 intrinsically resistant to tamoxifen and Herceptin) had IC50 and IC100 values of 20 nM and 375 nM, respectively. MCF-7 TAM1 (MCF-7 acquired resistance to tamoxifen) had IC50 and IC100 values of 25 nM and 200 nM, respectively. T47D had IC50 and IC100 values of 14 nM and 20 nM, respectively. All four cell lines also had a positive ER status as shown in Table 2, and each, as the values indicate, was AF-sensitive.

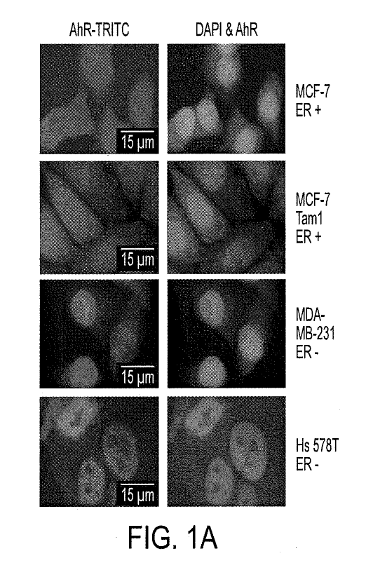

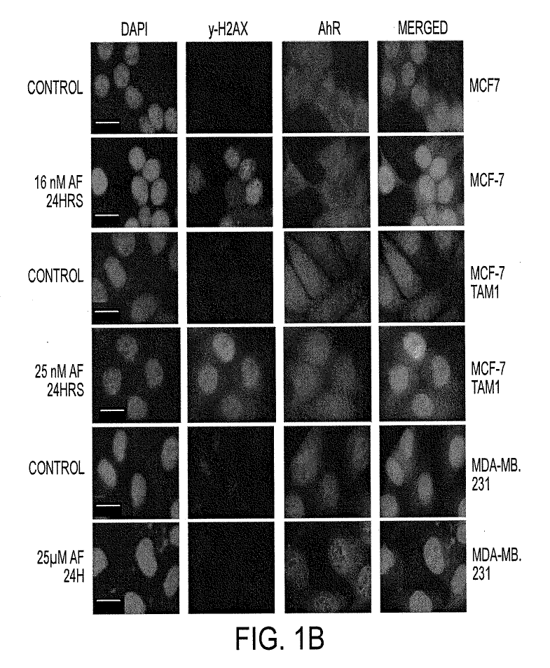

On the other hand, the MDA-MB-231 and MCF10A cell lines were deemed AF-resistant. MDA-MB-231 had IC50 and IC100 values of 25 μM and >100 μM, respectively. MCF10A had IC50 and IC100 values of 3 μM and 9 μM, respectively. Both of these cell lines had AhR localized predominantly in the nucleus. As shown in FIG. 1A, AhR is predominantly localized in the cytoplasm of AF-sensitive MCF-7 and MCF-TAM1 breast cancer cell lines. TRITC is used in the lefthand column to show the cytoplasm. DAPI is used in the righthand column to show the nucleus (by binding to DNA). Also shown in FIG. 1A is the predominant localization of AhR in the nucleus in AF-resistant MDA-MB-231 and Hs578T breast cancer cell lines.

FIG. 1B shows (in the second column) an immunofluorescence of phosphorylated γ-H2AX, a marker for DNA double strand breaks. γ-H2AX staining shows that the AF:AhR complex has translocated from the cytoplasm to the nucleus to activate a signaling cascade ultimately leading to apoptosis. The phosphorylated γ-H2AX is an indicator of a DNA-damage response in cells. Without being bound by theory, the localization of γ-H2AX indicates that AF:AhR complexes are responsible for DNA damage.

TABLE 2

| | Cell Line | | | ER | AhR | γ-H2AX |
|---|---|---|---|---|---|---|
| Cell Line | Characteristics | IC₅₀ | IC₁₀₀ | Status | Status | Foci |
| MCF-7 | breast cancer cell | 16 nM | 300 nM | positive | cytoplasmic | yes |
| | line | | | | | |
| | (adenocarcinoma) | | | | | |
| MCF-7 HER2-18 | MCF-7 intrinsically | 20 nM | 375 nM | positive | cytoplasmic | yes |
| | resistant to | | | | | |
| | tamoxifen and | | | | | |
| | Herceptin | | | | | |
| MCF-7 TAM1 | MCF-7 acquired | 25 nM | 200 nM | positive | cytoplasmic | yes |
| | resistance to | | | | | |
| | tamoxifen | | | | | |
| T47D | breast cancer cell | 14 nM | 20 nM | positive | cytoplasmic | yes |
| | line (infiltrating | | | | | |
| | ductal carcinoma) | | | | | |
| MDA-MB-231 | invasive breast | 25 μM | >100 μM | negative | nuclear | no |
| | cancer line | | | | | |
| | (adenocarcinoma) | | | | | |
| MCF10a | immortal, normal | 3 μM | 9 μM | negative | nuclear | no |
| | breast cell line | | | | | |

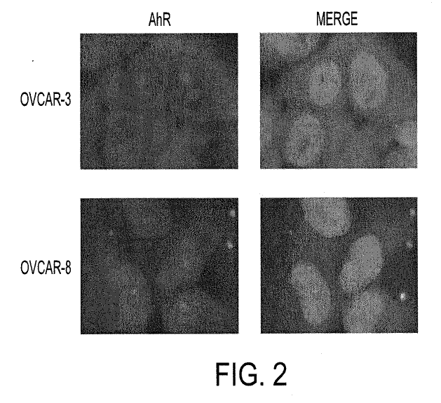

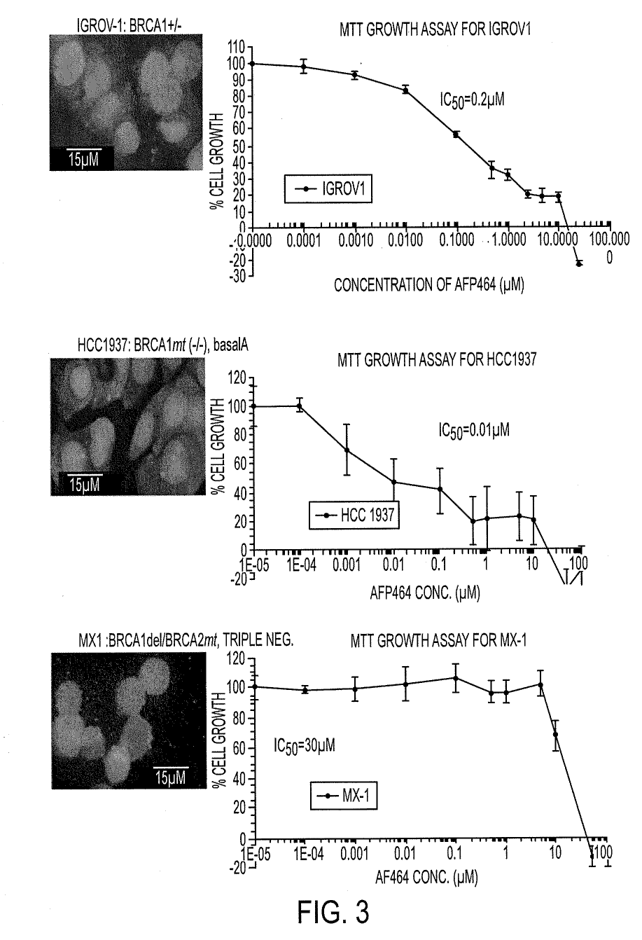

Similarly, the ovarian cancer cell lines shown in Table 3 were screened to determine the localization of the Aryl hydrocarbon receptor (AhR). As shown below, and further illustrated in FIGS. 2 and 3, all cell lines where the AhR is localized predominantly in the cytoplasm (e.g. OVCAR-3 and IGROV-1) were more sensitive to treatment with AFP464 than tumor cells where the AhR is localized predominantly in the nucleus (e.g., OVCAR-8). OVCAR-3 had an IC50 value of 0.252 μM. IGROV-1 had an IC50 value of 0.42 μM. AF-resistant OVCAR-8 had an IC value of 13 μM.

The localization of AhR, and its impact on determining whether the cell lines are AF-sensitive or AF-resistant can be valuable to determine a course of treatment. Further, the treatment of AFP464 can be extended to other types of tumors in which AhR is known to be predominantly localized in the cytoplasm, such as, for example, pancreatic tumors.

TABLE 3

| Cell Line | | |
|---|---|---|
| Ovarian | IC50 (μM) | AhR |
| OVCAR-8 | 13 | nuclear |
| ADDP | 2.5 | ND |
| AG6000 | >10 | ND |
| A2780 | 5 | ND |
| OVCAR-3 | 0.252 | cytoplasmic |
| IGROV-1 | 0.42 | cytoplasmic |

FIG. 3 shows the localization of AhR in the triple negative MX-1 breast cancer cell line, the ER-negative, basal A HCC1937 breast cancer cell line, and the IGROV1 ovarian cancer cell line. AhR localization correlates with cytotoxic activity of AFP464, as discussed above. MX-1 cells with nuclear AhR are resistant to treatment with AFP464 (IC50=30 uM), whereas the HCC1937 and IGROV1 lines with cytoplasmic AhR are sensitive to AFP464 (IC50s 10-200 nM). The growth curves for all three cell lines resulting from 5 day continuous exposure of the cells to AFP464 in 96-well plates and the detection of cell growth by conversion of methyltetrazolium into formazan by viable cells (MTT assay) are shown. All cell lines in FIG. 3 have defects in the BRCA tumor suppressor genes responsible for very aggressive forms of hereditary breast and ovarian cancer. MX-1 have BRCA1 deletions and BRCA2 mutations, HCC1937 are BRCA1 mutant and completely defective, IGROV1 are defective for BRCA2 (±).

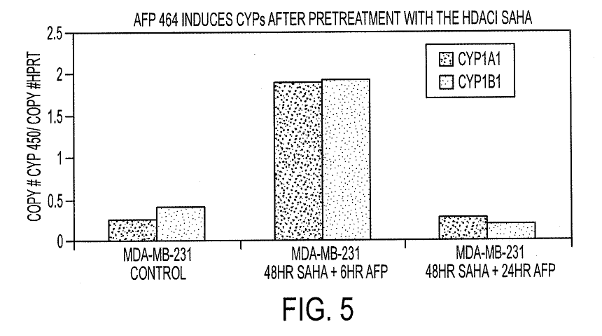

In sensitive cells, AFP464 induces AhR-mediated cytochrome P450 (CYP)-dependent xenobiotic response and cell death. In resistant cells, the CYP system is not induced. Real time PCR assessment showed the induction of CYP1A1 and CYP1B1 by treatment with HDAC inhibitors and AFP464 in MDA-MB-231 cells that AhR-dependent xenobiotic response was restored (FIG. 5, discussed further below).

As shown below in Table 4, the genetic profile of 11 breast cancer cell lines was determined and each cell line was tested to determine the 50% inhibitory concentration (IC50) of aminoflavone. The inhibitory concentration 50% were determined by MTT assay and following the NCI DTP in vitro testing procedures (http://dtp.nci.nih.gov/branches/btb/ivclsp.html). Three independent MTT in vitro tumor cell growth inhibition experiments were conducted and a mean IC50 value generated as shown in Table 4.

TABLE 4

| | Gene | | | | |
|---|---|---|---|---|---|
| Cell Line | Cluster | ER | PR | Her2 | AF IC50 (uM) |
| Hs578t | Basal B | − | − | − | 20 |
| MCF10A | Basal B | − | − | − | 3 |
| MDA-MB-231 | Basal B | − | − | − | 25 |
| HCC1937 | Basal A | − | − | − | 0.010 |
| BT 20 | Basal A | − | − | + | 0.020 |
| MDA-MB-468 | Basal A | − | − | − | 0.012 |
| SKBR3 | Luminal | − | − | +++ | 0.016 |
| T47D | Luminal | + | + | + | 0.014 |
| MCF-7 | Luminal | + | + | + | 0.016 |
| MCF-7 Tam1 | Luminal | + | + | + | 0.025 |
| MCF-7 Her2-18 | Luminal | + | + | +++ | 0.020 |

The SKBR3, T47D, MCF-7, MCF-7 Tam1, and MCF-7 Her2-18 cell lines are all of a luminal type gene cluster. These cell lines all have AF IC50 concentrations in the 0.016-0.020 micromolar range indicating sensitivity to AF464. Based on previous NCI cell line screen results discussed above, ER-negative tumors were thought to be resistant to aminoflavone. However, as shown in Table 4, HCC1937, BT 20, MDA-MB-468 (all of basal A type), and SKBR3 (luminal type), all are ER-negative tumor cells and all are sensitive to AFP464. The AF IC50 concentrations for these ER-negative cell lines range from as low as 0.010-0.020 micromolar. Thus, the gene profile relating to ER status alone may not predict the sensitivity of the tumor to treatment with AF. AFP464 is not only effective in ER-positive breast cancer cells, which are always of luminal type histologies, but are also effective in the basal A subtype of ER-negative breast cancers.

In another case, as shown in Tables 5A and 5B, pre-treatment of ER-negative, or “triple-negative,” or basal B breast cancer cell lines with a HDAC inhibitor, such as, for example, suberoylanalide hydroxamic acid (SAHA), modulates the ER status of the cell lines and makes the cell lines sensitive to aminoflavone with IC50 of ˜1 μM. These results indicate that sensitization of these cell lines by SAHA is time, schedule, and cell type dependent.

TABLE 5A
MDA-MB-231.

| | Exp. 1 | | | Exp. 3 | | | Exp. 4 | | |
|---|---|---|---|---|---|---|---|---|---|
| | ED50 | ED75 | ED90 | ED50 | ED75 | ED90 | ED50 | ED75 | ED90 |
| SAHA (24) + AFP464(96) | | | | | | | | | |
| AFP464 + SAHA | 1.08663 | 3.63316 | 12.44893 | 0.35438 | 1.00829 | 2.90052 | 1.52655 | 3.6797 | 9.92458 |
| SAHA(48) + AFP464 (72) | | | | | | | | | |
| AFP464 + SAHA | 0.07142 | 0.03236 | 0.01466 | 0.4279 | 0.47916 | 0.54527 | 0.18607 | 0.13101 | 0.09303 |
| SAHA (72) + AFP464 (48) | | | | | | | | | |
| SAHA + AFP464 | 0.92913 | 0.9087 | 0.88872 | 0.4375 | 0.8893 | 1.80919 | 0.6585 | 0.59213 | 0.53885 |

Numbers in parentheses are time in hours; ED, effective dose, numbers are combination indices.

TABLE 5B
Hs578T.

| | Exp. 1 | | | Exp. 2 | | |
|---|---|---|---|---|---|---|
| | ED50 | ED75 | ED90 | ED50 | ED75 | ED90 |
| SAHA (24) + AFP464(96) | | | | | | |
| AFP464 + SAHA | 0.37052 | 0.19258 | 0.10208 | 0.30101 | 0.1203 | 0.04813 |
| SAHA(48) + AFP464 (72) | | | | | | |
| AFP464 + SAHA | 1.3957 | 1.80861 | 2.34419 | 1.53713 | 2.1993 | 3.15448 |
| SAHA (72) + AFP464 (48) | | | | | | |
| SAHA + AFP464 | 1.43221 | 1.47419 | 1.52054 | 2.80212 | 4.10876 | 6.03168 |

Combination studies were performed according to the method by Chou and Talalay (see Chou, T. C., and Talalay, P. Quantitative analysis of dose-effect relationships: The combine effects of multiple drugs or enzyme inhibitors, Adv. Enzyme Regul., 22:27-55, 1984) at the fixed IC50 ratio by employing a range of 8 concentrations for each drug and determining growth by MTT assay. In one case, synergism of two agents exists if the combination indices at the effective (ED) doses 50, 75 or 90% are below 1, if they are 1, drugs are additive; above 1, drugs are antagonistic.

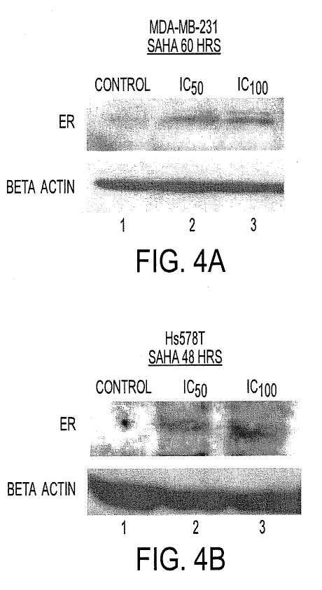

FIGS. 4A and 4B are Western blots showing induction of ERα in MDA-MB-231 (FIG. 4A) and Hs578T (FIG. 4B) breast cancer cell lines when treated with SAHA (in this case Vorinostat). Lane 1 of each of FIGS. 4A and 4B is a control in which the respective cells were treated with DMSO. Lanes 2 and 3 of FIG. 4A show MDA-MB-231 cells treated with SAHA for 60 hours at 2.5 μM (IC50) and 13.5 μM (IC100), respectively. As shown, the ERα induction increases after SAHA treatment. Lanes 2 and 3 of FIG. 4B show similar results in which the Hs578T breast cancer cell lines were treated with SAHA for 48 hours at 8 μM (IC50) and 100 μM (IC100), respectively.

FIG. 5 is a bar graph measuring the induction of the CYP1A1 and CYP1B1 pathways in MDA-MB-231 breast cancer cells after treatment with SAHA followed by treatment with AFP464. The bar graphs on the left are controls showing little expression of RNA for CYP1A1 and CYP1B1. The middle graphs show increased expression of RNA for CYP1A1 and CYP1B1 after treatment with SAHA for 48 hours followed by treatment with AFP464 for 6 hours. The graphs on the right show little expression of RNA for CYP1A1 and CYP1B1 after treatment with SAHA for 48 hours followed by treatment with AFP464 for 24 hours indicating that the sensitization by SAHA is time and schedule dependent. The immunofluorescent signals were normalized for copy number by using CYP1A1 and CYP1B1 expression vector constructs and by generating a standard curve with these vector cDNAs. Induction of CYP1A1 and CYP1B1 is seen 6 hrs after AFP464 treatment in SAHA pretreated cells compared to base line and 24 hrs AFP464.

In vivo studies expounding on the above discussed findings were then conducted. The MDA-MB-231 breast carcinoma cell line was originally established from a patient tumor at the MD-Anderson Cancer Center in Houston, Tex. These cells were obtained from the American Type Culture Collection (Manassas, Va.). The cell line belongs to the category of “triple negative” breast cancers, lacking estrogen and progesterone receptor as well s HER2/neu. The cell line also harbors a p53 mutation.

Thymus aplastic nude mice of Ncr/nu genetic background were used for establishment and serial propagation of the human tumor xenograft MDA-MB-23 1 from the cell line. Tumor fragments (size, ˜30 mm³) were implanted subcutaneous into nude mice and treatment was initiated when tumors reached a median volume of ˜70 mm³ (˜10 days after transplantation, early stage). This subcutaneous xenograft staging system has been defined by the U.S. National Cancer Institute's Drug Development Program. Tumors with a median volume of 190 mm³ (100-400 mm³) are termed advanced stage. A model is considered early stage if treatment is initiated when tumor sizes range from 63 to 200 mm³. See Alley M C, Hollingshead M G, Dykes D J, Waud W R. Human tumor xenograft models in NCI drug development. In: Teicher B A, Andrews P A, editors. Anticancer drug development guide: preclinical screening, clinical trials, and approval, 2nd ed. Totowa (N.J.): Humana Press, Inc.; 2004. p. 125-52. MDA-MB-231 is a fast growing tumor (average doubling time in log-growth ˜4.5 days) and has a >95% take rate, hence fulfilling National Cancer Institute criteria for a suitable early-stage tumor xenograft model. See Fiebig H H, Burger A M. Human tumor xenografts and explants. In: Teicher B A, editor. Animal models in cancer research. Totowa (N.J.): Humana Press, Inc.; 2001. p. 113-37; see also Geran R I, Greenberg N H, MacDonald M M, Schumacher A M, Abbott B J. Protocols for screening chemical agents and natural products against animal tumors and other biological systems. Cancer Chemother 1972; Rep. 3:1-103. A group contained 7 to 8 mice each with one to two tumors per flank.

Next, the treatment and data evaluation will be discussed. AFP464 was dissolved in 5% sterile glucose solution (vehicle) and given intravenously, and Vorinostat/SAHA was formulated in methylcellulose/Tween80 and given orally. The maximum tolerated dose (MTD) for AFP464 was determined prior to initiation of the experiment as 75 mg/kg/d intravenously. SAHA was administered orally at non toxic doses, of 50 mg/kg/d. The MTD for SAHA in mice is 175 mg/kg/day. In this experiment, SAHA administered 150 mg over three consecutive days (day -2, -1 and d0, d12-14). AFP464 was given on days 1, 3, 5, 15, 17, and 19.

Tumor growth was followed by serial caliper measurement, body weights recorded, and tumor volumes were calculated using the standard formula (length×width²)/2, where length is the largest dimension and width the smallest dimension perpendicular to the length. Whereas tumor volume in mm³is the appropriate variable deduced from this formula, it has to be noted that 1 mm³ equals 1 mg of tumor weight. Data were evaluated using the National Cancer Institute guidelines for assessment of anticancer drug effects in subcutaneously growing human tumor xenografts. Using specifically designed software (Study Director), the median relative tumor volume was plotted against time. Relative tumor volumes were calculated for each single tumor by dividing the tumor volume on day X by that on day 0 (time of randomization. Growth curves were analyzed in terms of maximal tumor inhibition/optimal % treated versus control (T/C) where changes in tumor volume (δTV) for each treated (T) and control (C) group were calculated for each day. Tumors were measured by subtracting the median tumor volume on day of first treatment (staging day) from the median tumor weight on the specified observation day. These values were used to calculate a % T/C as follows:

% T/C=(ΔT/ΔC)×100, where ΔT>0

or

% T/C=(ΔT/T_{1)×}100, where ΔT<0

and T₁=median tumor volume at start of the treatment. The optimum (minimum) value obtained is used to quantitate antitumor activity and the day at which this effect occurs is indicated. Tumor inhibition is defined as an optimal T/C that is <50. Partial tumor regressions are defined as tumor volume decreases to 50% of less of the tumor volume at the start of treatment, complete regressions are the instances in which the tumor burden decreases below 63 mm³ at any time during the experimental period.

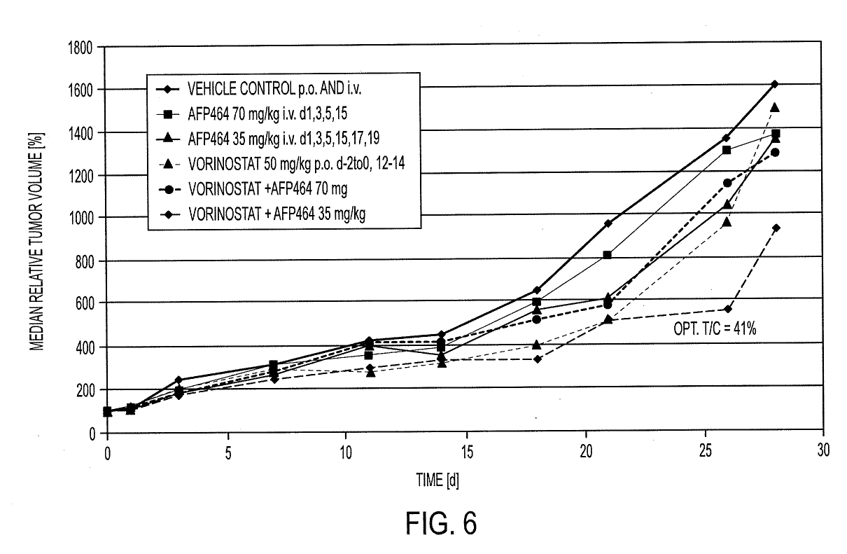

FIG. 6 and Table 6 show the results as well as the treatment schedule of the in vivo studies. The median relative tumor volume of the control group was, as expected, consistently greater than the other groups. The group that was unexpectedly lower than the other groups was that of SAHA+AFP464 35 mg/kg. These results show that pretreatment with SAHA and treatment with AF can increase the percent growth inhibition of tumors in otherwise resistant breast cancer.

TABLE 6

| | | | | | Median | Opt. | % Growth |
|---|---|---|---|---|---|---|---|
| | | Dosage | | | Starting Body | Test/Control | Inhibition |
| Grps | Compound | (mg/kg) | Frequency | Route | Wt. (gram) | % [d] | [d29] |
| 1 | Vehicle (Glucose, | 10 mll/kg | Day −3 to −1, 12-14 | p.o. | 24.66 | 100 | — |
| | Tween80, | | Day 1, 3, 5, 15, 17, 19 | i.v. | | | |
| | Methylcellulose) | | | | | | |
| 2 | AFP464 | 70 | Day 1, 3, 5, 15 | i.v. | 24.15 | 85 [d28] | 15 |
| 3 | AFP464 | 35 | Day 1, 3, 5, 15, 17, 19 | i.v. | 24.93 | 77 [d26] | 23 |
| 4 | Vorinostat | 50 | Day −3 to −1, 12-14 | p.o. | 24.36 | 71 [d26] | 29 |
| 5 | AFP464 − Vorinostat | 70/50 | Day 1, 3, 5, 15 | i.v. | 24.30 | 80 [d28] | 20 |
| | | | Day −3 to −1, 12-14 | p.o. | | | |
| 6 | AFP464 + Vorinostat | 35/50 | Day 1, 3, 5, 15, 17, 19 | i.v. | 24.00 | 41 [d26] | 59 |
| | | | Day −3 to −1, 12-14 | p.o. | | | |

Treatment of a cancer patient is discussed below. A biopsy of a tumor can be taken from a patient. The biopsy can be analyzed by histological techniques and/or gene clusters can be analyzed as described herein. If the patient has ER-positive breast cancer, aminoflavone compounds are administered to the patient to achieve a concentration of about 1 μM in patient plasma. If the patient has breast cancer that is resistant to hormonal therapy, aminoflavone compounds are administered to the patient. If the patient has breast cancer that is resistant to both hormonal and Herceptin therapy, aminoflavone compounds are administered to the patient.

The gene profile of breast tumor is determined, and aminoflavone compounds are administered to the patient if the tumor shows luminal or basal A types of gene cluster. If the patient has breast cancer with either ER-negative or “triple-negative”, or basal B type of gene cluster, the patient is pre-treated with a histone deacetylase (HDAC) inhibitor before treatment with aminoflavone compounds. Pre-treatment with a HDAC inhibitor modulates the estrogen receptor signaling and makes the cells sensitive to aminoflavone.

In other instances, the localization of the aryl hydrocarbon receptor (AhR) in a tumor (e.g. breast, ovarian, renal or pancreatic cancer) is determined and if the tumor has AhR predominantly localized to the cytoplasm, aminoflavone compounds are administered to the patient.

Alternatively or in addition to the above, a patient having failed hormonal treatment may be treated with single-agent AFP464 74 mg/m² administered intravenously on days 1 and 8 (D1 and D8) of a 21 day cycle. Optionally, patients who had failed up to 2 prior chemotherapeutic regimens may be treated with SAHA 400 mg/d PO for 5 days prior to each intravenously administered AFP464 dose at 74 mg/m² on D1 and D8 of a 21 day cycle. Treatment may be given until disease progression or untolerable toxicity. AFP464 may be administered as a 3 hour intravenously infusion at 74 mg/m². Vorinostat may be administered orally five days prior to each AFP464 dose.

The above description and drawings illustrate the disclosure herein. Those skilled in the art will recognize that substitutions, additions, deletions, modifications and/or other changes may be made to the above disclosure.

We claim:

1. A method for reducing breast tumor volume, comprising:
determining if the breast tumor comprises cells that are of a type selected from the group consisting of estrogen receptor-negative luminal subtype gene cluster and a basal A subtype gene cluster; and
administering an effective amount of a compound to a mammal having the breast tumor if the breast tissue sample comprises cells of a type selected from the group consisting of an estrogen receptor-negative luminal subtype gene cluster and a basal A subtype gene cluster, the compound having general formula: 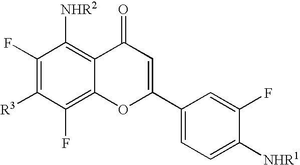
wherein each of R¹ and R² is H, COCH₂—R⁷, wherein R⁷ is amino, branched or straight-chain alkylamino, dialkylamino, or alkyl- or dialkylaminoalkyl, or an a-amino acid residue, provided that at least one of R¹ and R² is other than H;
wherein R³ is H, branched or straight-chain alkyl, hydroxyalkyl, alkanoyloxyalkyl, alkanoyloxy, alkoxy, or alkoxyalkyl, or a pharmaceutically acceptable salt thereof; and
wherein the volume of the breast tumor is reduced by at least about 15%.

2. The method of claim 1, wherein the compound is:

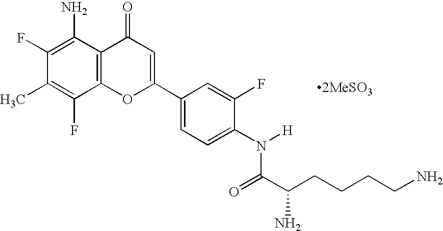

3. The method of claim 1, wherein the compound is selected from the group consisting of 5-amino-6,8-difluoro-2-[3-fluoro-4-[(L-lysyl)amino)]phenyl]-7-methyl-4H-1-benzopyran-4-one, 5-amino-2-[4-[2-amino-5-guanidinopentanoyl]amino]-3-fluorophenyl]-6,8-difluoro-7-methyl-4H-1-benzopyran-4-one, 6,8-difluoro-7-methyl-5-(dimethylamino)acetamido-2-[4-(dimethylamino)acetamido-3-fluorophenyl]-4H-1-1-benzopyran-4-one, and 5-amino-6,8-difluoro-7-methyl-2-[4-(dimethylamino)acetamido-3-fluorophenyl ]-4H-1-benzopyran-4-one.

4. The method of claim 1, wherein the compound is administered orally, parenteraly, or topically.

5. The method of claim 4, wherein the parenteral administration is selected from the group consisting of intravenous, intraperitoneal, intrapulmonary, or intrathecal.

6. The method of claim 1, wherein the step of determining the cell type comprises performing a gene expression profile.

7. The method of claim 6, wherein the step of determining the cell type comprises determining whether ERBB-3 and ESR-1 genes in the cells of the breast tissue sample are overexpressed.

8. The method of claim 7, wherein the overexpression of the ERBB-3 or the EST-1 gene in the breast tissue sample is at least about 2-fold higher than the expression of the ERBB-3 or the EST-1 gene in non-tumorigenic breast tissue.

9. The method of claim 6, wherein the step of determining the cell type comprises determining whether KRT5- and KRT14-genes in the cells of the breast tissue sample are overexpressed.

10. The method of claim 9, wherein the overexpression of the KRT5- or KRT14-genes is at least about 2-fold greater than the expression of the KRT5- or KRT14-genes in non-tumorigenic breast tissue.

11. A method for inhibiting the growth of a tumor in a patient in need of treatment, comprising:
administering a histone deacetylase (HDAC) inhibitor to the patient; and
subsequently administering to the patient a compound having the following general formula: 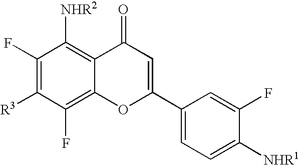
wherein each of R¹ and R² is H, COCH₂—R⁷, wherein R⁷ is amino, branched or straight-chain alkylamino, dialkylamino, or alkyl- or dialkylaminoalkyl, or an a-amino acid residue, provided that at least one of R¹ and R² is other than H;
wherein R³ is H, branched or straight-chain alkyl, hydroxyalkyl, alkanoyloxyalkyl, alkanoyloxy, alkoxy, or alkoxyalkyl, or a pharmaceutically acceptable salt thereof; and
wherein the growth of the tumor is reduced by about 15 to about 85%.

12. The method of claim 11, wherein the compound is:

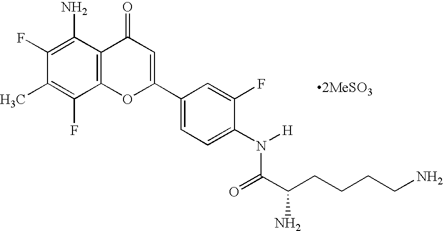

13. The method of claim 11, wherein the compound is selected from the group consisting of 5-amino-6,8-difluoro-2-[3-fluoro-4-[(L-lysyl)amino)]phenyl]-7-methyl-4H-1-benzopyran-4-one, 5-amino-2-[4-[2-amino-5-guanidinopentanoyl]amino]-3-fluorophenyl]-6,8-difluoro-7-methyl-4H-1-benzopyran-4-one, 6,8-difluoro-7-methyl-5-(dimethylamino)acetamido-2-[4-(dimethylamino)acetamido-3-fluorophenyl]-4H-1-benzopyran-4-one, and 5-amino-6,8-difluoro-7-methyl-2-[4-(dimethylamino)acetamido-3-fluorophenyl ]-4H-1-benzopyran-4-one.

14. The method of claim 11, wherein administering the treating compound to the patient results in a concentration of about 0.1 μM to about 500 μM of the growth inhibiting compound in plasma of the patient.

15. The method of claim 11, wherein the HDAC inhibitor is suberoylanilide hydroxamic acid (SAHA).

16. The method of claim 11, wherein the HDAC inhibitor is administered 2 to 7 days prior to administration with the growth inhibiting compound.

17. The method of claim 16, wherein the HDAC inhibitor is administered 3 to 6 days prior to administration with the growth inhibiting compound.

18. The method of claim 17, wherein the HDAC inhibitor is administered 5 days prior to administration with the growth inhibiting compound.

19. The method of claim 18, wherein the HDAC inhibitor is administered orally.

20. The method of claim 19, wherein the HDAC inhibitor is administered at a dose of about 1 to about 100 mg/kg.

21. The method of claim 20, wherein the HDAC inhibitor is administered at a dose of about 25 to about 75 mg/kg.

22. The method of claim 21, wherein the HDAC inhibitor is administered at a dose of about 50 mg/kg.

23. The method of claim 11, wherein the compound is AFP464.

24. The method of claim 11, wherein the compound is administered at a dose of about 1 to about 100 mg/kg.

25. The method of claim 24, wherein the compound is administered at a dose of about 35 to about 70 mg/kg.

26. The method of claim 25, wherein the compound is administered at a dose of about 35 mg/kg.

27. The method of claim 11, further comprising administering a second dose of a HDAC inhibitor after administration of the compound.

28. The method of claim 27, wherein the HDAC inhibitor is administered 10 to 15 days after the administration of the growth inhibiting compound.

29. The method of claim 28, wherein the HDAC inhibitor is administered 12 to 14 days after the administration of the growth inhibiting compound.

30. The method of claim 29, wherein the HDAC inhibitor is administered successively on each day 12 to 14 days after the administration of the growth inhibiting compound.

31. The method of claim 27, further comprising administering at least one additional dose of the compound after the second dose of the HDAC inhibitor.

32. The method of claim 31, wherein the at least one additional dose of the compound is administered 3 days after the second dose of the HDAC inhibitor.

33. The method of claim 32, wherein at least one additional dose of the compound is administered at a dose of about 35 mg/kg.
